(19) United States
(12) Patent Application Publication
(10) Pub. No.: US 2022/0019981 A1
(43) Pub. Date: Jan. 20, 2022

(54) ONLINE IN-PERSON MARKETPLACE

(71) Applicant: TYMEYT Corporation, Farmington Hills, MI (US)

(72) Inventors: Joydeep Bhattacharya, Farmington Hills, MI (US); Rumeli Das, Farmington Hills, MI (US); Abhinandan Bhattacharya, Farmington Hills, MI (US)

(21) Appl. No.: 16/932,783

(22) Filed: Jul. 19, 2020

(51) Int. Cl. G06Q 10/10; G06Q 30/02; G16H 50/80; G06Q 50/26

(52) U.S. Cl. CPC G06Q 10/1095; G06Q 30/0201; G06Q 50/26; G16H 50/80; G06Q 30/0277

(57) ABSTRACT

A system and method for providing online customer-business connection and scheduling is disclosed. The system is configured to communicatively interconnect a web server, and one or more client devices, over the Internet, The web server includes a memory having instructions stored thereon and a processor configured to execute the instructions on the memory to cause the web server to determine a usable capacity for a business for scheduling in-person appointments for providing goods and services to a customer, the usable capacity being a regional percentage set by an appropriate entity of a based upon current conditions in the region, accept an appointment request for a client appointment from a customer using one of the client devices, provide a set of available appointment times to the one of the customer in response to the appointment request, accept a selected appointment time from the set of available appointment times from the customer, schedule the selected appointment time for the customer to meet in-person with the business to conduct commerce, and provide reminders of the selected appointment to the customer and the business before the selected appointment.

### TECHNICAL FIELD

This application relates in general to a system and method for providing online commerce, and more specifically, to a system and method for providing online customer-business connection and scheduling by implementing Social Distancing requirement embedded into the process.

### BACKGROUND

Currently, the entire world is maintaining indefinite lock-down with governments generally implementing stay-at-home orders for citizens in an effort to minimize social interaction of the population during the COVID-19 pandemic. As a result, customers are typically staying at home and businesses in need of customers are mostly closed or possibly open with very limited capacity. Customers do not know when it may be appropriate to attempt to visit a business such as a barber shop because they lack information about whether the business is open and whether the business has the ability and capacity to interact with them should they arrive. Similarly, businesses do not know if or when a customer may arrive and how many customers may arrive at any particular time. Therefore, it is difficult for all parties to know a safe and proper way to attempt to engage in the purchase/sale of goods and services while maintaining social distancing between all individuals.

Local rules and conditions may limit the time, conditions, and number of individuals who may meet to complete transactions for goods and services. One locale may have a high density of COVID19 infections, so in post lock-down that jurisdiction may be permitted to utilize one percentage of each business's actual full-capacity at any point of time. At the same time, a different jurisdiction may permit a larger percentage of a business's actual full-capacity because the level of infections in that locale is lower. Over time, the permitted operating rules in each of these jurisdictions may dynamically change as the level of infections rise and fall. All of these circumstances make it difficult for both businesses and customers to attempt to engage in commerce that require in-person interactions between the businesses and customers while remaining safe and complying with all of the relevant rules, regulations, and governmental orders.

Therefore, a need exists for both these parties to connect through a unified online marketplace application for scheduling while complying with all applicable rules, regulations, and governmental orders. Use of such a system may guide customers regarding when to go to a particular business and when it may be appropriate to delay/avoid a visit at a given point in time. Use of such a system, at the same time, assists businesses to foresee their business estimation in advance which will help them to plan better in maintaining the supply chain. By collecting data associated with these transactions may also aid governmental efforts to perform contact tracing as members of the population interact to engage in ordinary commerce. The present invention attempts to address all of the above deficiencies in existing systems according to the functions and principles disclosed herein.

### SUMMARY

In accordance with the present invention, the above and other problems are solved by providing a system and method for online customer-business connection and scheduling according to the principles and example embodiments disclosed herein.

In one embodiment, the present invention is a system for providing online customer-business connection and scheduling. The system is configured to communicatively interconnect a web server, and one or more client devices, over the Internet, The web server includes a memory having instructions stored thereon and a processor configured to execute the instructions on the memory to cause the web server to determine a usable capacity for a business for scheduling in-person appointments for providing goods and services to a customer, the usable capacity being a regional percentage set by an appropriate entity based upon current conditions in the region, accept an appointment request for a client appointment from a customer using one of the client devices, provide a set of available appointment times to the one of the customer in response to the appointment request, accept a selected appointment time from the set of available appointment times from the customer, schedule the selected appointment time for the customer to meet in-person with the business to conduct commerce, and provide reminders of the selected appointment to the customer and the business before the selected appointment.

In another embodiment, the present invention is a method for providing online customer-business connection and scheduling using an online system configured to communicatively interconnect a web server, and one or more client devices, over the Internet. The method determines a usable capacity for a business for scheduling in-person appointments for providing goods and services to a customer, the usable capacity being a regional percentage set by an appropriate entity of a ç based upon current conditions in the region, accepts an appointment request for a client appointment from a customer using one of the client devices, provides a set of available appointment times to the one of the customer in response to the appointment request, accepts a selected appointment time from the set of available appointment times from the customer, schedules the selected appointment time for the customer to meet in-person with the business to conduct commerce, and provides reminders of the selected appointment to the customer and the business before the selected appointment.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of a system that provides online customer-business connection and scheduling according to the present invention.

FIG. 2a is a block diagram illustrating an exemplary hardware architecture of a computing device.

FIG. 2b is a block diagram illustrating an exemplary logical architecture for a client device.

FIG. 2c is a block diagram showing an exemplary architectural arrangement of clients, servers, and external services.

FIG. 2d is another block diagram illustrating an exemplary hardware architecture of a computing device.

FIG. 3 illustrates a flowchart corresponding to a method performed by software components providing an initial party interaction with online customer-business connection and scheduling according to the present invention.

FIG. 4 illustrates a flowchart corresponding to a method performed by software components that provides party registration to permit them to utilize the online customer-business connection and scheduling system according to the present invention.

FIG. 5 illustrates a flowchart corresponding to a method performed by software components that provides a registered business access to the online customer-business connection and scheduling system according to the present invention.

FIG. 6 illustrates a flowchart corresponding to a method performed by software components that provides a registered customer access to the online customer-business connection and scheduling system according to the present invention.

FIG. 7 illustrates another example embodiment of a system for providing online customer-business connection and scheduling according to the present invention.

### DETAILED DESCRIPTION

This application relates in general to a system and method for providing online commerce, and more specifically, to a system and method for providing online customer-business connection and scheduling according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The term “mobile application” refers to an application executing on a mobile device such as a smartphone, tablet, and/or web browser on any computing device.

The terms “customer,” “client,” and “user” refer to an entity, e.g. a human, using the Online In-Person Marketplace System including any software or smart device application(s) associated with the invention. The term user herein refers to one or more users.

The term “connection” refers to connecting any component as defined below by any means, including but not limited to, a wired connection(s) using any type of wire or cable for example, including but not limited to, coaxial cable(s), fiberoptic cable(s), and ethernet cable(s) or wireless connection(s) using any type of frequency/frequencies or radio wave(s). Some examples are included below in this application.

The term “invention” or “present invention” refers to the invention being applied for via the patent application with the title “Online In-Person Marketplace System.” Invention may be used interchangeably with online marketplace.

The terms “communicate” or “communication” refer to any component(s) connecting with any other component(s) in any combination for the purpose of the connected components to communicate and/or transfer data to and from any components and/or control any settings.

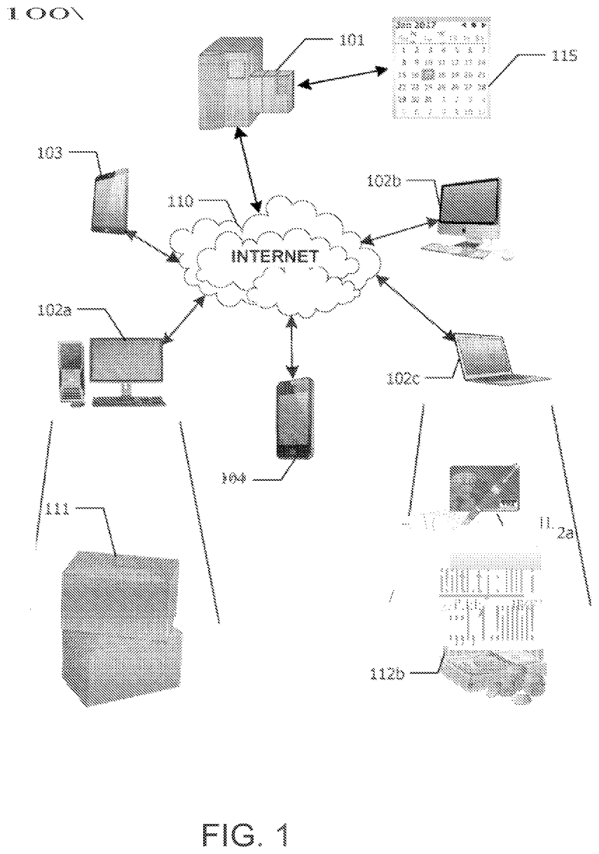

In general, the present disclosure relates to a system and method for providing online commerce. To better understand the present invention, FIG. 1 illustrates an example embodiment for a system 100 for online customer-business connection and scheduling according to the present invention. An online-in-person marketplace system 100 provides a centrally-managed online marketplace for any type of customers and/or businesses to avail themselves and/or offer services with a built-in capacity-utilization based intelligent social distancing mechanism to fight regular and/or pandemic situations specific to particular geography.

The system 100 connects a merchant offering goods or services 111 for purchase 112a-b to a customer. These parties may utilize many different computing devices 102a-n, mobile tablets 103, and smartphones 104 to communicate with a central marketplace server 101 to connect a merchant and customer, to enable the transaction to occur including online payment, and to schedule a date and time 115 in which the customer may be physically present with the merchant to complete a transaction.

The system 100 creates an online marketplace for customers, businesses, and facilities permitting them to book appointments to purchase merchandise, schedule services, place orders, complete payment, provide feedback, share a review, and utilize coupons while maintaining an appropriate social distance for both merchants and customers and while assisting businesses to utilize limited/full capacity depending on pandemic intensity in that geographic area. At the same time, this system 100 may also sets appropriate access conditions for business users such as their available capacity and available bookings using appropriate conditions set by responsible governmental authorities for a given locale where the business is located. The system may also provide COVID-19 infection data tracing information to local government or health authorities on a demand/need basis. Users may opt-in or opt-out of participation in the contract tracing services as desired.

For example, the system 101 may dynamically change the capacity of the commerce provided by a business user with a time gap in between two consecutive customer services to meet current conditions for a locale. Unless walk in customers are also permitted, business/customer will be allowed to perform their in-store business/service seamlessly ensuring social distancing and minimal overlap of touches/over crowd situation.

In order for this result to be realized, a few steps are followed. At the time of registration, business users are asked to the system 101 with their full normal capacity as to what the business user may serve at a time. This value may be stated as X number of people. When the system 101 receives an API call related to COVID data, the system sets the available utilization for the business to an allowable limit is 60% utilization. The system also requests business users to provide a measure of the total floor area available to the business user. The system 101 uses a formula: π*radius squared, where radius is 6 feet to determine the amount of customers may be simultaneously safely services at a given time. Therefore, the business user's total available area is divided by A=113.097336 ft2 will determine a maximum number of customers allowed. Take a conservative choice between above two figures and take the average of those two to allow final number of concurrent customer users for a business. This value is used by the system 101 when providing potential customers with possible appointment times to visit the business in question.

The present invention may use any type of network such as a single network, multiple networks of a same type, or multiple networks of different types which may include one or more of a direct connection between devices, including but not limited to a local area network (LAN), a wide area network (WAN) (for example, the Internet), a metropolitan area network (MAN), a wireless network (for example, a general packet radio service (GPRS) network), a long term evolution (LTE) network, a telephone network (for example, a Public Switched Telephone Network or a cellular network), a subset of the Internet, an ad hoc network, a fiber optic network (for example, a fiber optic service (often known as FiOS) network), or any combination of the above networks.

Smart devices mentioned herein the present application may also use one or more sensors to receive or send signals, such as wireless signals for example, Bluetooth™, wireless fidelity, infrared, Wi-Fi, or LTE. Any smart device mentioned in this application may be connected to any other component or smart device via wired communications (e.g., conductive wire, coaxial cable, fiber optic cable, ethernet cable, twisted pair cable, transmission line, waveguide, etc.), or a combination of wired and wireless communications. The invention's method and/or system may use a single server device or a collection of multiple server devices and/or computer systems.

The systems and methods described above, may be implemented in many different forms of applications, software, firmware, and hardware. The actual software or smart device application codes or specialized control software, hardware or smart device application(s) used to implement the invention's systems and methods is not limiting of the implementation. Thus, the operation and behavior of the systems and methods were described without reference to the specific software or firmware code. Software, smart device application(s), firmware, and control hardware can be designed to implement the systems and methods based on the description herein.

This new invention also has the ability to manage, control or communicate with multiple or unlimited number of billboards from one or more server or computer system location without the intervention of the operator or operators or anyone with access or privileges to use this new invention. For example, one or more billboards can be managed or controlled or communicated with one or more servers, computer systems, or smart devices from one or more locations. To further exemplify, the user will be able to control or communicate with as many billboards as desired from one centralized location if desired or more than one location.

While all of the above functions are described to be provided to users via a mobile application on a smartphone, one of ordinary skill will recognize that any computing device including tablets, laptops, and general purpose computing devices may be used as well. In at least one embodiment, all of the services described herein are provided using web pages being accessed from the web server 201 using a web browser such as Safari™, Firefox™, Chrome™ DuckDuckGo™, and the like. All of the screen examples described herein show user interface elements that provide the functionality of the present invention. The arrangement, organization, presentation, and use of particular user input/output (I/O) elements including hyperlinks, buttons, text fields, scrolling lists, and similar I/O elements are shown herein for example embodiments only to more easily convey the features of the present invention. The scope of the present invention should not be interpreted as being limited by any of these elements unless expressly recited within the attached claims.

For the purposes of the example embodiment of FIG. 1, various functions are shown to be performed on different programmable computing devices that communicate with each other over the Internet 105. These computing devices may include smartphones 101a, laptop computers 101b, tablets (not shown), and similar devices so long as the disclosed functionality of the mobile application described herein is supported by the particular computing device. One of ordinary skill will recognize that this functionality is grouped as shown in the embodiment for clarity of description. Two or more of the processing functions may be combined onto a single processing machine. Additionally, it may be possible to move a subset of processing from one of the processing systems shown here and retain the functionality of the present invention. The attached claims recite any required combination of functionality onto a single machine, if required, and all example embodiments are for descriptive purposes.

For all of the above devices that are in communication with each other, some or all of them need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects, and in order to more fully illustrate one or more aspects. Similarly, although process steps, method steps, algorithms or the like may be described in a sequential order, such processes, methods, and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method or algorithm is carried out or executed. Some steps may be omitted in some aspect or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the aspects disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific aspects, at least some of the features or functionalities of the various aspects disclosed herein may be implemented on one or more general-purpose computers associated with one or more networks, such as for example, an end-user computer system, a client computer, a network server or other server system, a mobile computing device (e.g., tablet computing device, mobile phone, smartphone, laptop or other appropriate computing device), a consumer electronic device, a music player or any other suitable electronic device, router, switch or other suitable device, or any combination thereof. In at least some aspects, at least some of the features or functionalities of the various aspects disclosed herein may be implemented in one or more virtualized computing environments (e.g., network computing clouds, virtual machines hosted on one or more physical computing machines or other appropriate virtual environments).

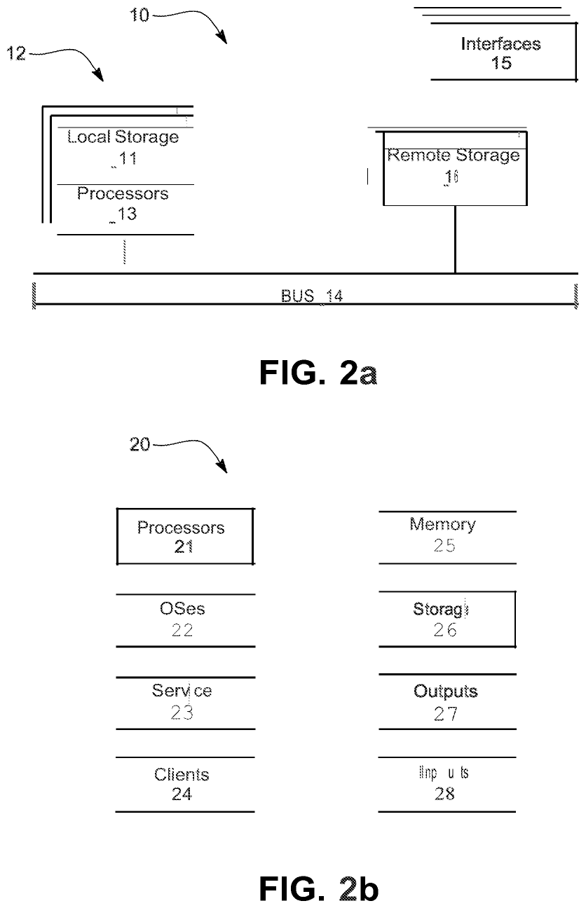

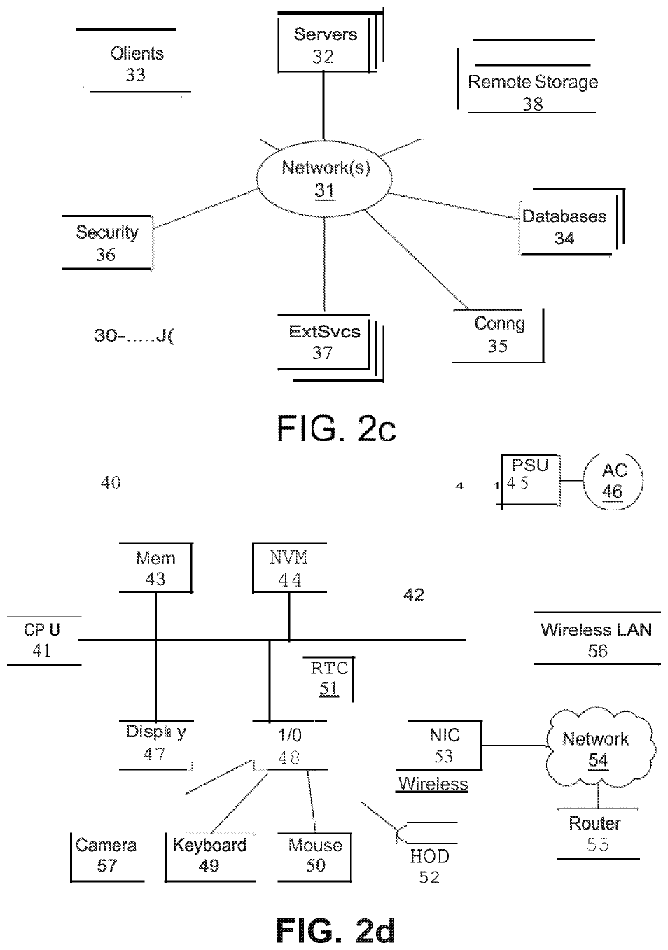

Referring now to FIG. 2a, there is a block diagram depicting an exemplary computing device 10 suitable for implementing at least a portion of the features or functionalities disclosed herein. A computing device 10 may be, for example, any one of the computing machines listed in the previous paragraph, or indeed any other electronic device capable of executing software- or hardware-based instructions according to one or more programs stored in memory. A computing device 10 may be configured to communicate with a plurality of other computing devices, such as clients or servers, over communications networks such as a wide area network, a metropolitan area network, a local area network, a wireless network, the Internet or any other network, using known protocols for such communication, whether wireless or wired.

In one aspect, a computing device 10 includes one or more central processing units (CPU) 12, one or more interfaces 15, and one or more buses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing a CPU 12, local memory 11 and/or remote memory 16, and interface(s) 15. In at least one aspect, a CPU 12 may be caused to perform one or more of the different types of functions and/or operations under the control of software modules or components, which for example, may include an operating system and any appropriate applications software, drivers, and the like.

A CPU 12 may include one or more processors 13 such as for example, a processor from one of the Intel, ARM, Qualcomm, and AMD families of microprocessors. In some aspect, processors 13 may include specially designed hardware such as application-specific integrated circuits (ASICs), electrically erasable programmable read-only memories (EEPROMs), field-programmable gate arrays (FPGAs), and so forth, for controlling operations of a computing device 10. In a particular aspect, a local memory 11 (such as non-volatile random access memory (RAM) and/or read-only memory (ROM), including for example, one or more levels of cached memory) may also form part of a CPU 12. However, there are many different ways in which memory may be coupled to a system 10. Memory 11 may be used for a variety of purposes such as, for example, caching and/or storing data, programming instructions, and the like. It should be further appreciated that a CPU 12 may be one of a variety of system-on-a-chip-(SOC) type hardware that may include additional hardware such as memory or graphic processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may, for example, support other peripherals used with a computing device 10. Among the interfaces that may be provided are ethernet interfaces, frame relay interfaces, cable interfaces, DSL interfaces, token ring interfaces, graphics interfaces, and the like. In addition, various types of interfaces may be provided such as, for example, universal serial bus (USB), serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast ethernet interfaces, gigabit ethernet interfaces, serial ATA (SATA) or external SATA (ESATA) interfaces, high-definition multimedia interfaces (HDMI), digital visual interfaces (DVI), analog or digital audio interfaces, asynchronous transfer mode (ATM) interfaces, high-speed serial interfaces (HSSI), point of sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 2a illustrates one specific architecture for a computing device 10 for implementing one or more of the aspects described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, a single processor 13 handles communications as well as routing computations, while in other aspects a separate dedicated communications processor may be provided. In various aspects, different types of features or functionalities may be implemented in a system according to the aspect that includes a client device (such as a tablet device or smartphone running client software) and a server system (such as a server system described in more detail below).

Regardless of network device configuration, the system of an aspect may employ one or more memories or memory modules (for example, remote memory block 16 and local memory 11) configured to store data, program instructions for the general-purpose network operations or other information relating to the functionality of the aspects described herein (or any combinations of the above). Program instructions may control execution of or comprise an operating system and/or one or more applications, for example. Memory 16 or memories 11, 16 may also be configured to store data structures, configuration data, encryption data, historical system operations information or any other specific or generic non-program information described herein.

Because such information and program instructions may be employed to implement one or more systems or methods described herein, at least some network device aspects may include non-transitory machine-readable storage media, which, for example, may be configured or designed to store program instructions, state information, and the like for performing various operations described herein. Examples of such non-transitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device) or they may be removable such as swappable flash memory modules (such as “thumb drives” or other removable media designed for rapidly exchanging physical storage devices), “hot-swappable” hard disk drives or solid state drives, removable optical storage disks, or other such removable media, and that such integral and removable storage media may be utilized interchangeably. Examples of program instructions include both object code, such as may be produced by a compiler, machine code, such as may be produced by an assembler or a linker, byte code, such as may be generated by for example by a JAVA™ compiler and may be executed using a JAVA™ virtual machine or equivalent, or files containing higher level code that may be executed by the computer using an interpreter (for example, scripts written in Python™, Perl™, Ruby™, Groovy™, or any other scripting language).

In some aspects, systems may be implemented on a standalone computing system. Referring now to FIG. 2b, there is a block diagram depicting a typical exemplary architecture of one or more aspects or components thereof on a standalone computing system. A computing device 20 includes processors 21 that may run software that carry out one or more functions or applications of aspects, such as for example a client application 24. Processors 21 may carry out computing instructions under control of an operating system 22 such as, for example, a version of MICROSOFT WINDOWS™ operating system, APPLE macOS™ or iOS™ operating systems, some variety of the LINUX™ operating system, ANDROID™ operating system, or the like. In many cases, one or more shared services 23 may be operable in system 20, and may be useful for providing common services to client applications 24. Services 23 may, for example, be WINDOWS™ services, user-space common services in a LINUX™ environment or any other type of common service architecture used with an operating system 21. Input devices 28 may be of any type suitable for receiving user input including, for example, a keyboard, touchscreen, microphone (for example, for voice input), mouse, touchpad, trackball or any combination thereof. Output devices 27 may be of any type suitable for providing output to one or more users, whether remote or local to system 20, and may include, for example, one or more screens for visual output, speakers, printers or any combination thereof. Memory 25 may be RAM having any structure and architecture known in the art for use by processors 21, for example to run software. Storage devices 26 may be any magnetic, optical, mechanical, memristor or electrical storage device for storage of data in digital form (such as those described above, referring to FIG. 2a). Examples of storage devices 26 include flash memory, magnetic hard drive, CD-ROM, and the like.

In some aspects, systems may be implemented on a distributed computing network, such as one having any number of clients and/or servers. Referring now to FIG. 2c, there is a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in Fig. B. In addition, any number of servers 32 may be provided for handling requests received from one or more clients 33. Clients 33 and servers 32 may communicate with one another via one or more electronic networks 31, which may be in various aspects any Internet, wide area network, mobile telephony network (such as CDMA or GSM cellular networks), wireless network (such as WiFi, WiMAX, LTE, and so forth) or local area network (or indeed any network topology known in the art; the aspect does not prefer any one network topology over another). Networks 31 may be implemented using any known network protocols, including, for example, wired and/or wireless protocols.

In addition, in some aspects, servers 32 may call external services 37 when needed to obtain additional information, or to refer to additional data concerning a particular call. Communications with external services 37 may take place, for example, via one or more networks 31. In various aspects, external services 37 may comprise web-enabled services or functionality related to or installed on the hardware device itself. For example, in one aspect where client applications 24 are implemented on a smartphone or other electronic device, client applications 24 may obtain information stored on a server system 32 in the Cloud or on an external service 37 deployed on one or more of a particular enterprise's or user's premises. In addition to local storage on servers 32, remote storage 38 may be accessible through the network(s) 31.

In some aspects, clients 33 or servers 32 (or both) may make use of one or more specialized services or appliances that may be deployed locally or remotely across one or more networks 31. For example, one or more databases 34 in either local or remote storage 38 may be used or referred to by one or more aspects. It should be understood by one having ordinary skill in the art that databases in storage 34 may be arranged in a wide variety of architectures and use a wide variety of data access and manipulation means. For example, in various aspects one or more databases in storage 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some aspects, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system or a logical database within an overall database management system. Unless a specific meaning is specified for a given use of the term “database,” it should be construed to mean any of these senses of the word, all of which are understood as a plain meaning of the term “database” by those having ordinary skill in the art.

Similarly, some aspects may make use of one or more security systems 36 and configuration systems 35. Security and configuration management are common information technology (IT) and web functions, and some amount of each are generally associated with any IT or web system. It should be understood by one having ordinary skill in the art that any configuration or security subsystems known in the art now or in the future may be used in conjunction with aspects without limitation, unless a specific security 36 or configuration system 35 or approach is required by the description of any specific aspect.

FIG. 2d shows an exemplary overview of a computer system 40 as may be used in any of the various locations throughout the system. It is exemplary of any computer that may execute code to process data. Various modifications and changes may be made to a computer system 40 without departing from the broader scope of the system and method disclosed herein. A CPU 41 is connected to bus 42, to which bus is also connected to memory 43, nonvolatile memory 44, display 47, I/O unit 48, and network interface card (NIC) 53. An I/O unit 48 may, typically, be connected to peripherals such as a keyboard 49, pointing device 50, hard disk 52, real-time clock 51, camera 57, and other peripheral devices. A NIC 53 connects to a network 54, which may be the Internet or a local network, which local network may or may not have connections to the Internet. The system may be connected to other computing devices through the network via a router 55, wireless local area network 56 or any other network connection. Also shown as part of a system 40 is a power supply unit 45 connected, in this example, to a main alternating current (AC) supply 46. Not shown are batteries that could be present and many other devices and modifications that are well known, but are not applicable to, the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles or other integrated hardware devices).

In various aspects, functionality for implementing systems or methods of various aspects may be distributed among any number of client and/or server components. For example, various software modules may be implemented for performing various functions in connection with the system of any particular aspect, and such modules may be implemented to run on server and/or client components.

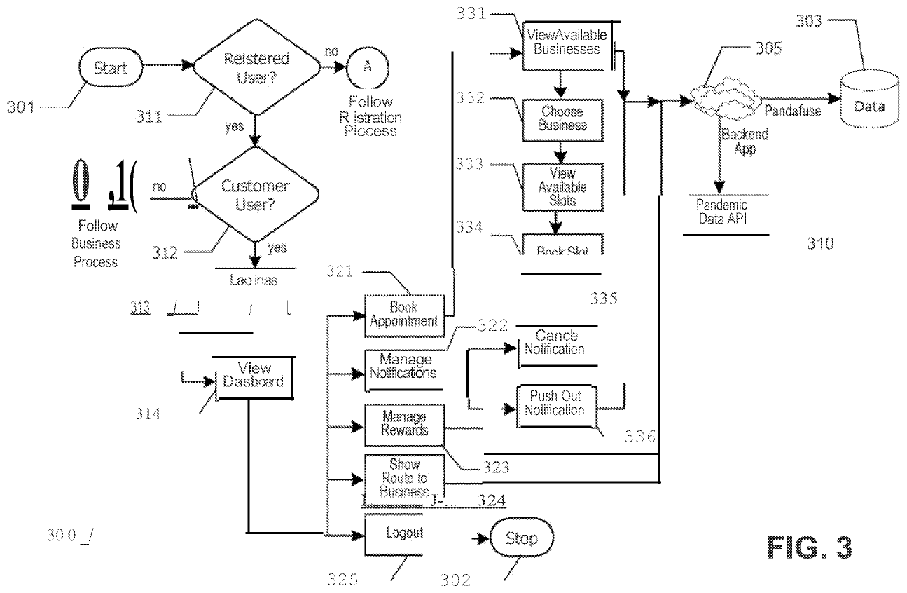

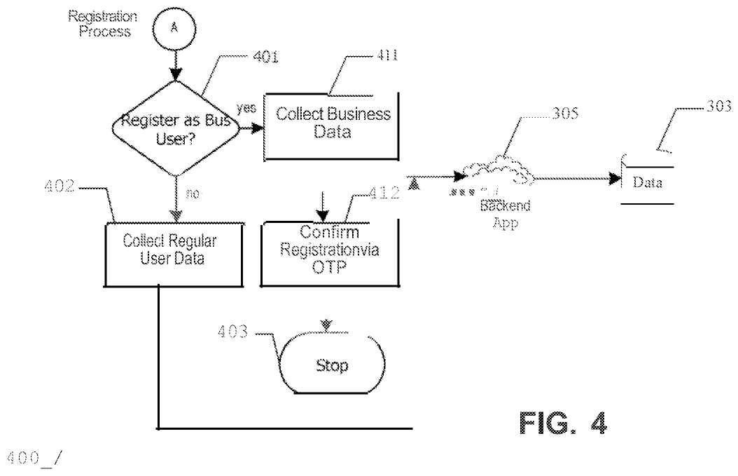

FIG. 3 illustrates a flowchart corresponding to a method performed by software components providing an initial party interaction with an online customer-business connection and scheduling system according to the present invention. The process begins 301 and a test step 311 determines whether the user is a registered user and if not, the process follows A to FIG. 4 to perform the user registration process.

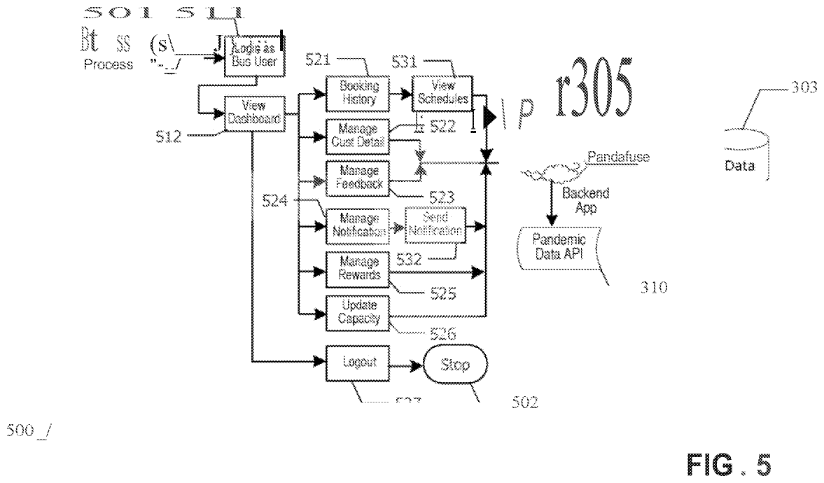

When the test step 311 determines that the user is registered, test step 312 determines whether the user is a customer user, and when not the process follow B to FIG. 5 to perform business related actions. When test step 312 determines that the user is a customer, the user logs into the system 100 in step 313 and views a customer dashboard providing one or more actions the customer may take in step 314. From the customer dashboard, the customer may book an appointment in step 321, manage notifications in step 322, manage rewards in step 323, show a travel route to a business in step 324, and log out in step 325. The book an appointment step 321 begins a process permitting a customer to schedule a time to go to a business to complete a purchase of merchandise or a service. The customer views all available businesses of a particular type capable of providing the merchandise or service in step 331. The customer, in step 332, selects a business to provide the merchandise or service and views any and all available time slots on one or more dates in step 333. The customer books the appointment in step 334 and the appointment is processed by a backend application 305 on the server 101 to store the data into a data store 303.

When the customer selects the manage notifications step 322 from the customer dashboard, notifications of appointments for the customer may be managed. The customer may cancel notification of the appointment in step 335 and may push a notification out for triggering a reminder in step 336. The notification change in both steps is then processed by the backend application 305 on the server 101 to store the data into a data store 303

When the customer selects the manage rewards step 323, the customer may manage a frequent or returning customer reward program associated with the customer account. There will be a Reward Center in the mobile app for both General User and Business User where they will see all their rewards earned/coupons etc. Rewards can be earned by various ways, and these can be redeemed after it crosses a formidable threshold. This module will be linked with payment gateway too. The changes in user data associated with the rewards program are processed by a backend application 305 on the server 101 to store the data into a data store 303.

When the customer selects the show a travel route to a business in step 324, a travel route is generated from the customer's location to the business. The travel route used in step 324 may obtain a current customer location from a web browser on a computing device 102a-n or from location data generated within a mobile application using Wifi or GPS data sources. The business location used in step 324 may be obtained from customer input or from a business address associated with a scheduled appointment. The routing function of step 324 may generate the fastest route measured in travel time, the cheapest route measured in gas and toll expenses, and the most relaxing route that avoids road construction, traffic congestion, and weather conditions. The generated route may be processed by a backend application 305 on the server 101 to store the data into a data store 303 to permit retrieval of the route taken by the customer.

The log out step 325 ends 302 all processing.

FIG. 4 illustrates a flowchart corresponding to a method performed by software components that provides party registration to permit users to utilize the online customer-business connection and scheduling system according to the present invention. The user registration process begins with A that originated in FIG. 3. Test step 401 determines whether the new user is a business user, and if not, step 402 collects regular customer data for use in the customer's user profile. The system 100 confirms the customer contact information in step 412 with the use of a one-time password (OTP) that is sent by the system to the user for immediate submission to the server 101. The customer user profile data may be processed by a backend application 305 on the server 101 to store the data into a data store 303.

When test step 401 determines the new user is a business user, step 411 collects business user data for use in the business user's profile. The system 100 confirms the business' contact information in step 412 with the use of a one-time password (OTP) that is sent by the system to the business user for immediate submission to the server 101. The business user profile data may be processed by a backend application 305 on the server 101 to store the data into a data store 303. Once the new user accounts are verified using the OTP, the process ends 403.

FIG. 5 illustrates a flowchart corresponding to a method performed by software components that provides a registered business access to the online customer-business connection and scheduling system according to the present invention. The registered business process begins with B that originated in FIG. 3 and the business user logs into the system 100 in step 511 and views a business user dashboard providing one or more actions the business user may take in step 512. From the business user dashboard, the business user may manage its booking history in step 521, manage customer details in step 522, manage customer feedback in step 523, manage notifications in step 324, manage rewards in step 325, manage capacity in step 526, and log out in step 527. When the business user selects the booking history step 521, the business user may view present, past, and future appointments, cancel present and future appointments, and modify present and future appointments in step 521. Once changes are made, the business user may view a current schedule for appointments in step 531. Changes to the booking schedule are processed by a backend application 305 on the server 101 to store the data into a data store 303.

When the business user selects the manage customer details step 522, the business user may view and use customer profile data associated with an appointment in step 522. All uses of the customer profile data are processed by a backend application 305 on the server 101 to store the data into a data store 303.

When the business user selects the manage customer feedback step 523, the business user may view and respond to all customer feedback following an appointment booking and following completion of a transaction for goods or services. All responses to feedback are processed by a backend application 305 on the server 101 to store the data into a data store 303.

When the business user selects the manage notifications step 324, notifications of appointments scheduled for the business user may be managed. The business user may cancel notification of the appointment and may push a notification out for triggering a reminder. The change in notification is sent to the customer in step 532. The notification change is then processed by a backend application 305 on the server 101 to store the data into a data store 303.

When the business user selects the manage rewards step 325, the business user may manage a frequent or returning customer reward program associated with the goods or services provided by the business user associated with the business user account. Changes to the rewards program are processed by a backend application 305 on the server 101 to store the data into a data store 303.

When the business user selects the manage capacity step 526, the business user may manage available capacity to schedule appointments with customers in step 526. The business user may add and reduce current capacity of the business and may add and reduce future capacity of the business as of a specified date and time. All changes to the business capacity are processed by a backend application 305 on the server 101 to store the data into a data store 303.

When the business user selects the log out step 527 the process stops 502.

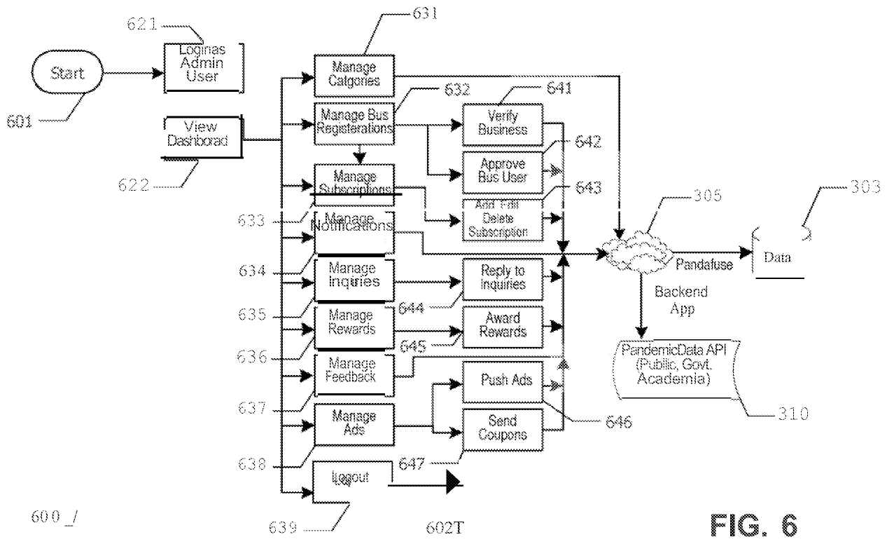

FIG. 6 illustrates a flowchart corresponding to a method performed by software components that provides a registered administrator access to the online customer-business connection and scheduling system according to the present invention. The registered administrator process begins 601 and the admin user logs into the system 100 in step 621 and views an admin user dashboard providing one or more actions the administrator may take in step 622. From the admin user dashboard, the administrator may manage business goods and services categories in step 631, manage business user registrations in step 632, manage subscriptions in step 633, manage notifications in step 634, manage inquiries in step 635, manage rewards in step 636, manage feedback in step 637, manage advertisements in step 638, and log out in step 639.

When the admin user selects the manage business goods and services categories step 631, the administrator may add, modify and delete categories of goods and services used in classifying business user offerings within the system 100. All changes to the business goods and services categories are processed by a backend application 305 on the server 101 to store the data into a data store 303.

When the admin user selects the manage business user registrations step 632, the administrator verifies a business user profile data in step 641, The administrator also approves business users for offering goods and services on the system 100 in step 642. All changes to the business user registration submissions are processed by a backend application 305 on the server 101 to store the data into a data store 303.

When the admin user selects the manage subscriptions step 633, the administrator may define, modify and delete various subscriptions available for users to purchase. Subscription will be for both a Customer, a general user, and a Business, a commerce user, with different monthly/annual plans, which can be set/altered from Admin Panel. All changes to the subscriptions are processed by a backend application 305 on the server 101 to store the data into a data store 303.

When the admin user selects the manage notifications step 634, notifications of appointments scheduled for the admin user may be managed. The administrator may view, set and cancel notifications associated with an appointment. The notification change is then processed by a backend application 305 on the server 101 to store the data into a data store 303.

When the admin user selects the manage inquiries step 635, the administrator may view and respond to all inquiries received from potential customers and potential businesses in step 644. All inquiry replies are processed by a backend application 305 on the server 101 to store the data into a data store 303.

When the admin user selects the manage rewards step 636, the administrator may manage a frequent or returning customer reward program associated with the goods or services provided by one or more business users. The administrator may award rewards to customers in step 645. Changes to the rewards program and its data are processed by a backend application 305 on the server 101 to store the data into a data store 303.

When the admin user selects the manage feedback step 637, the administrator may view and respond to all customer feedback following an appointment booking and following completion of a transaction for goods or services. All responses to feedback are processed by a backend application 305 on the server 101 to store the data into a data store 303

When the administrator user selects the manage advertisements step 638, the administrator may create, modify and delete advertisements that may appear when users visit the marketplace. These advertisements provide a hyperlink to the business in question for a customer to schedule an appointment. All changes to the business capacity are processed by a backend application 305 on the server 101 to store the data into a data store 303.

When the admin user selects the log out step 639, the process stops 602.

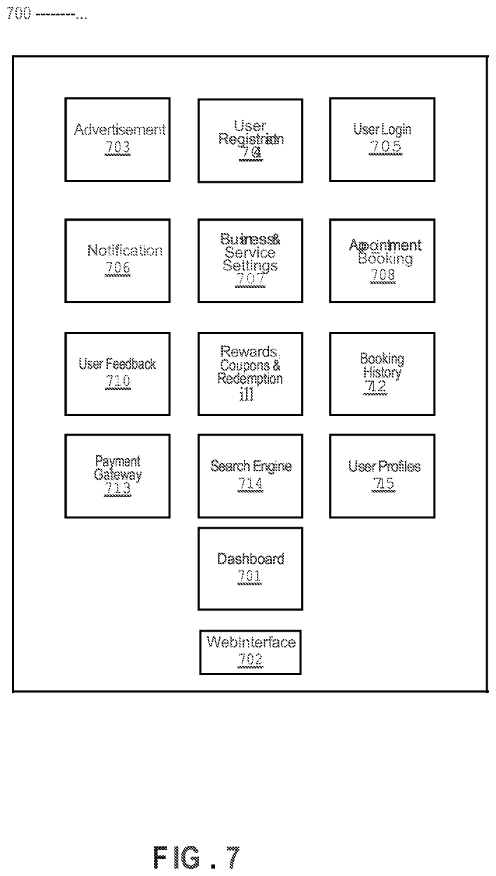

FIG. 7 illustrates another example embodiment of a system for providing an online customer-business connection and scheduling system according to the present invention. The web server 700 comprises a user dashboard 701, a web interface 702, an advertisement module 703, a user registration module 704, a user login 705, a notification manager 706, a business-services settings 707, an appointment booker 708, a user feedback module 710, a rewards, coupons, and redemption module 711, a booking history 712, a payment gateway 713, a search engine 714, and a set of user profiles 715.

The user dashboard 701 presents a user with a set of choices for the customer using the customer dashboard 622 and a set of choices for the business user using the business dashboard 512. Depending upon input from these users, the system 700 directs the processing to the appropriate processing element as discussed above in reference to FIGS. 3-6.

The web interface 702 connects the server 700 to the Internet 110 to send and receive communication from users, businesses, and the like. The web interface 702 performs all necessary data formatting, data packet creation, data encryption for security, and data transmission and reception when the server 700 communicates with other processing systems disclosed herein. The network interface 702 is also responsible to ensure reception of any communications to other computing systems and to log any errors or attempts to hack into the datastore.

The advertisement module 703 permits an administrator may create, modify and delete advertisements that may appear when users visit the marketplace. These advertisements provide a hyperlink to the business in question for a customer to schedule an appointment.

The user registration module 704 permits both customer users and business users to register creating an account to use the system 700. The processing for creation of an account is described above in reference to FIG. 4.

The user login 705 permits users to connect to and access the system 700. The user login 705 is responsible for authenticating a user based upon user input. Typically, the user input uses a username and password. Multi-factor authentication, use of one-time passwords, and similar secure authentication mechanisms may be included in the user login 705. For every sign in system will recognize the user type, i.e. buyer or seller, along with all past activities from account details in database. Based on user type, the system 700 behavior will change.

The notification manager 706 provides users with notification of an upcoming event that has been scheduled. This notification may appear as messages and other indications on a notification panel when the user logs into the system 700. The event notifier 706 may also send messages to the user via other communication mechanisms such as email, text messages and the like if contact information for the user is captured and stored in the user profile for use by the event notifier 706. Administrator users may be able to send notification from the notification manager 706 to a targeted user group as defined therein.

The business-services settings 707 permits registered business user to set their business and service from the given list with execution time for each service. The backend system 305 checks and puts restriction on maximum allowable seats, number of booking and the like as set for a locale in which the business is located to maintain social distancing effectively.

The appointment booker 708 permits user to schedule instore appointments. Customer users have an option to select a particular business type for scheduling from groups assigned and set by the administrators and business owners. Every selection navigate buyers to a relevant business page with booking option. Customer users select a date to check availability. Backend algorithm will provide available date and necessary time slots for the service by maintaining core concept of social distancing

The user feedback module 710 captures customer user feedback at various points in the booking and sale cycle. The user feedback module 710 utilizes a Feedback form to capture user experience. Once captured, an administrator user is notified by email. The administrator user may respond as necessary to the feedback from customer users.

The rewards, coupons, and redemption module 711 permits user to redeem coupons assigned to them based on points collected while using the system. Different reward packages and coupons may be defined by administrator users and provided to various defined groups of customer users. The payment gateway 713 is linked to this module to permit users to have coupons applied to their purchases when checking out a purchase.

The booking history 712 provides users with access to a listing of all past and pending appointments. Once a booking cycle is initiated, booking info get updated in booking history page with open status. After getting service user (both business owners and buyers) can mark that booking status as closed or failed with reason. Updating booking status may be incentivized with points.

The payment gateway 713 provides a mechanism for customer users to complete a transaction that has been booked using the system 700. For all transactions, the customer users are presented with an internal payment page to collect payment information such as credit card of bank payment information, account information for payment services such as Apple Pay™ PayPal™, Google Pay™, and the like. Once payment done through the selected payment gateway, a payment status is returned as a success or a failure. The respective database entry is updated as needed.

The search engine 714 assists customer users in searching business and services available on the system 700. Search engine 714 may be implemented as a separate layer top of appointment booker 708 and is data of available businesses having available time slots. The search engine 714 navigates user to booking pages for business users offering goods and services using system 700.

The set of user profiles 715 provides other users with information on the other users in the system. All user information is presented from this profile page. The profile page is how business users present their available goods and services to customer users using the system 700. Other user related items are performed through the user's profile page; for example, password changing, business page update, personal info update will be done through this page.

The embodiments described herein are implemented as logical operations performed by a computer. The logical operations of these various embodiments of the present invention are implemented (1) as a sequence of computer-implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise. Any singular term used in this present patent application is applicable to its plural form even if the singular form of any term is used.

In the present application, all or any part of the invention's software or application(s) or smart device application(s) may be installed on any of the user's or administrator's smart device(s), any server(s) or computer system(s) or web application(s) required to allow communication, transfer of content(s) or data between any combination of the components.

1. (canceled)

2. A system for providing online customer-business connection and scheduling, the system configured to communicatively interconnect a web server, and one or more client devices, over the Internet, the web server comprising:
a memory having instructions stored thereon; and
a processor configured to execute the instructions on the memory to cause the web server to:
determine a usable capacity for a business for scheduling in-person appointments for providing goods and services to a customer, the usable capacity being a regional percentage set by an appropriate entity of a region based upon current conditions in the region;
accept an appointment request for a client appointment from a customer using one of the client devices;
provide a set of available appointment times to the one of the customer in response to the appointment request;
accept a selected appointment time from the set of available appointment times from the customer;
schedule the selected appointment time for the customer to meet in-person with the business to conduct commerce;
provide reminders of the selected appointment to the customer and the business before the selected appointment;
register the business on to the web server to offer goods and services to the customer;
register the customer on the web server to search for business to conduct offered commerce, the customer provides a name, an address, and contact information; and
adjust the usable capacity of the business when the appropriate entity changes the regional percentage due to a change in conditions within the locale in which the business is located.

3. The system according to claim 2, wherein a total capacity of the business is determined using a measure of a total floor area of the business divided to provide a distance of at least 6 feet between customers.

4. The system according to claim 3, wherein the instructions on the memory to further cause the web server to:
offer rewards to customers;
provide directions for customers to the business;
accept feedback from customers; and
provide advertisements to customers when searching the web server for businesses.

5. The system according to claim 4, wherein the instructions on the memory to further cause the web server to:
maintain a database of customers and business employees present during appointments after the appointments occur; and
provide access to the database of customers and business employees to an appropriate entity to perform contact tracing of individuals present in a particular appointment.

6. (canceled)

7. A method for providing online customer-business connection and scheduling using an online system configured to communicatively interconnect a web server, and one or more client devices, over the Internet, the method comprising:
determining a usable capacity for a business for scheduling in-person appointments for providing goods and services to a customer, the usable capacity being a regional percentage set by an appropriate entity of a region based upon current conditions in the region;
accepting an appointment request for a client appointment from a customer using one of the client devices;
providing a set of available appointment times to the one of the customer in response to the appointment request;
accepting a selected appointment time from the set of available appointment times from the customer;
scheduling the selected appointment time for the customer to meet in-person with the business to conduct commerce;
providing reminders of the selected appointment to the customer and the business before the selected appointment;
registering the business on to the web server to offer goods and services to the customer;
registering the customer on the web server to search for business to conduct offered commerce, the customer provides a name, an address, and contact information; and
adjusting the usable capacity of the business when the appropriate entity changes the regional percentage due to a change in conditions within the locale in which the business is located.

8. The method according to claim 7, wherein a total capacity of the business is determined using a measure of a total floor area of the business divided to provide a distance of at least 6 feet between customers.

9. The method according to claim 8, wherein the method further comprises:
offering rewards to customers;
providing directions for customers to the business;
accepting feedback from customers; and
providing advertisements to customers when searching the web server for businesses.

10. The method according to claim 9, wherein the method further comprises:
maintaining a database of customers and business employees present during appointments after the ointments occur; and
providing access to the database of customers and business employees to an appropriate entity to perform contact tracing of individuals present in a particular appointment.
